(19) United States
(12) Patent Application Publication
(10) Pub. No.: US 2007/0071691 A1
(43) Pub. Date: Mar. 29, 2007

(54) Composition

(75) Inventors: Andrew Bruce Brown, RESEARCH TRIANGLE PARK, NC (US); Michiel Mary Van Oort, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: GLAXO GROUP LIMITED, RESEARCH TRIANGLE PARK, NC (US)

(21) Appl. No.: 10/576,461

(22) PCT Filed: Oct. 22, 2004
(86) PCT No.: PCT/GB04/04492

§ 371 Date: Aug. 16, 2006

(30) Foreign Application Priority Data
Oct. 24, 2003 (GB) 0324918.2

(51) Int. Cl. A61K 9/14

(52) U.S. Cl. 424/46

(57) ABSTRACT

Dry powder pharmaceutical compositions having improved storage stability, dry powder inhalers comprising the same and their use in the treatment of respiratory disorders by inhalation.

This invention relates to dry powder pharmaceutical compositions, and their use in the treatment of respiratory disorders by inhalation. The invention also relates to dry powder inhalers comprising the same. More particularly, this invention relates to dry powder pharmaceutical compositions having improved fine particle dose performance and/or improved stability.

### BACKGROUND OF THE INVENTION

Dry powder inhalers (DPI's) are well known devices for administering pharmaceutically active agents to the respiratory tract. Consequently, they are particularly suitable when used for the administration of active agents in the treatment of diseases such as asthma, bronchitis, chronic obstructive pulmonary disease (COPD), emphysema, rhinitis etc. Since the drug acts directly on the target organ much smaller quantities of the active ingredient may be used, thereby minimising any potential side effects.

Dry powder compositions for use as inhalable medicaments in DPI's typically comprise a pharmaceutically active agent intimately admixed with an excess of pharmaceutically acceptable excipient or excipients (often called carrier(s)). Such excipients serve not only to dilute the quantity of active agent administered in each dose but also to establish acceptable manufacture of the powder mixture and aid in the aerosolisation of the drug. Such a high proportion of excipient will essentially determine the properties of the powder formulation, particularly the manufacturing characteristics.

For effective delivery into the lungs, the active agent particles should be small, typically with a geometric diameter in the range of from 0.1 to 5 μm, or else an equivalent aerodynamic diameter substantially in the range of from 0.1 to 5 μm. However, small particles tend to aggregate with each other and/or with excipient particles, due to their high surface area to volume ratio, which provides excess surface free energy and encourages agglomeration.

It can thus occur that active agent particles, despite having a suitable particle size, do not reach the lung because they are attached to large excipient particles or to other particles of active agent. The fine particle dose (FPD), of drug is a measure of the quantity of drug of effectively deliverable particle size present in a released dosage of drug after actuation of the DPI. In some instances the FPD is referred to as the Fine particle mass (FPM), the terms to be taken as identical in meaning. Often it is convenient to refer to the fine particle fraction FPF, the % of the emitted dose that the fine particle dose represents. A high FPF is an indicator that a high portion of the administered drug will reach the lower lungs, where it can be effective. For a constant initial load of drug, the FPF is effectively equivalent to the FPD. In WO96/23485 (Coordinated Drug Development Limited) it is suggested that release of small particulate active from large excipient particles can be promoted (and thus the FPF increased) by the presence of an additive material on the surface of the excipient particles. There remains, however, a demand for further, or alternative, ways to increase the FPF.

It has now surprisingly been found that dry powder pharmaceutical compositions containing calcium stearate have surprisingly improved FPD/FPF properties. Such compositions represent an alternative solution to the above-noted problem.

### DESCRIPTION OF THE INVENTION

Dry powder pharmaceutical compositions for inhalation therapy comprising calcium stearate are believed to be novel. The present invention therefore provides, in a first aspect, a dry powder pharmaceutical composition for inhalation therapy comprising a pharmaceutically active agent, an excipient and calciumstearate. The invention also provides the use of calcium stearate in dry powder pharmaceutical compositions for inhalation therapy in order to increase FPD. In some circumstances, the excipient may be calcium stearate, such that the invention provides a dry powder pharmaceutical composition for inhalation therapy comprising a pharmaceutically active agent and calcium stearate.

A further problem associated with the use of dry powder pharmaceutical compositions of this type is that they can be susceptible to poor stability performance due to moisture ingress. For example, significant deterioration in the FPD/FPF, is often observed upon protracted exposure of such compositions to conditions of elevated temperature and humidity.

Patent application WO 00/28979 (SkyePharma) describes one approach to overcoming the above noted problems. It is claimed that dry powder formulations comprising a pharmaceutically active agent, an inhaled vehicle of non-inhalable particle size and magnesium stearate have improved storage stability under extreme (temperature and humidity) conditions.

We have now discovered that dry powder pharmaceutical compositions containing calcium stearate demonstrate surprisingly enhanced stability performance. Such compositions therefore represent an alternative solution to the above-noted problem.

The present invention therefore provides, in a second aspect, the use of calcium stearate in dry powder pharmaceutical compositions for inhalation therapy in order to improve stability performance.

The invention also provides for the use of calcium stearate in dry powder pharmaceutical compositions for inhalation therapy in order to eliminate or reduce the detrimental effect on fine particle dose caused by storage of said compositions.

The calcium stearate is preferably present in particulate form. The calcium stearate can be in amorphous or crystalline form. Preferably the calcium stearate is in crystalline form.

It is to be understood that the dry powder pharmaceutical compositions according to this invention include not only those in which the components are incorporated as individual particles but also those including matrix particles of more than one component. For example, matrix particles of pharmaceutically active agent and calcium stearate or matrix particles of excipient and calcium stearate may be utilised. Such matrix particles can be prepared by solid dispersion technology e.g. co-precipitation and particle coating methods which are familiar to those skilled in the art. Suitably, the components are incorporated as individual particles.

The term “calcium stearate” as used herein includes calcium stearate of various grades of purity. Stearic acid and calcium stearate as available commercially typically comprise a significant proportion of C₁₆ and C₂₀ fatty acid groups as well as the C₁₈ stearate.

Typically, the geometric size of the calcium stearate is in the range from 0.1 to 50 μm, and more particularly from 1 to 20 μm. Alternatively (depending on the density of the particles), the aerodynamic diameter of the particles is in the range from 0.1 to 50 μm, and more particularly from 1 to 20 μm. The material may be used as supplied. Alternatively, the calcium stearate for use in the preparation of compositions in accordance with this invention may have its particle size controlled. Optionally, the particles may be micronised but controlled precipitation, supercritical fluid methodology and spray drying techniques familiar to those skilled in the art may also be utilised. The calcium stearate may be present in a concentration of 0.01-99% by weight of the total composition. Suitably the calcium stearate is present in a concentration of 0.01-50% by weight of the total composition, preferably 1-20%, more preferably from 1 to 10%.

The pharmaceutically active agent can be any therapeutic molecule in dry powder form that is suitable to be administered by inhalation. In the field of inhalation therapy, the term “suitable to be administered by inhalation” is generally taken to mean therapeutic molecules having an aerodynamic diameter between 0.1 and 10 μm, and more particularly 1-5 μm. Particles of the desired particle size for inhalation are conventionally prepared by micronisation. Other methods of producing such particles are also known in the art. Therefore, such particles can also be prepared using controlled precipitation methods (e.g. methods described in patent applications WO 00/38811 and WO 01/32125 (Glaxo Group Limited)), using supercritical fluid methodology or by spray drying techniques. The present invention provides no limitation on the method by which the therapeutic molecule is made suitable to be administered by inhalation.

Examples of pharmaceutical active agents suitable for inhalation therapy include analgesics, e.g., codeine, dihydromorphine, ergotamine, fentanyl or morphine; anginal preparations, e.g., diltiazem; anti-allergics, e.g., cromoglycate (e.g. as the sodium salt), ketotifen or nedocromil (e.g. as the sodium salt); anti-infectives e.g., cephalosporins, penicillins, streptomycin, sulphonamides, tetracyclines and pentamidine; anti-histamines, e.g., methapyrilene or loratadine; anti-inflammatories, e.g., beclomethasone (e.g. as the dipropionate ester), fluticasone (e.g. as the propionate ester), flunisolide, budesonide, rofleponide, mometasone (e.g. as the furoate ester), ciclesonide, triamcinolone (e.g. as the acetonide), 6α, 9α-difluoro-11β-hydroxy-16α-methyl-3-oxo-17α-propionyloxy-androsta-1,4-diene-17β-carbothioic acid S-(2-oxo-tetrahydro-furan-3-yl)ester (also named as 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester) or 6α, 9α-Difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester; anti-tussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; PDE4 inhibitors e.g. cilomilast or roflumilast; leukotriene antagonists eg montelukast, praniukast and zafirlukast; adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate); iNOS inhibitors; α₄ integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy)acetyl]amino}pentanoyl)amino]propanoic acid (e.g. as free acid or potassium salt)], diuretics, e.g., amiloride; anticholinergics, e.g., ipratropium (e.g. as bromide), tiotropium, atropine or oxitropium; ganglionic stimulants, e.g., nicotine; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

Further suitable pharmaceutically acceptable agents include compounds known in the art as long acting β₂-adrenoreceptor agonists, particularly those generically and specifically described in patent applications WO 01/42183, WO 02/066422, WO 02/070490, WO 02/076933, WO 03/024439, WO 03/042160, WO 03/072539, WO 03/091204, WO 04/016578, WO 04/022547, WO 04/037807, WO 04/037772, WO 04/037768, WO 04/0379762 and WO 04/039766 Particularly preferred long acting β₂-adrenoreceptor agonists include:
3-(4-{[6-({(2R)-2-hydroxy-2-[4-hydroxy-3-(hydroxymethyl)-phenyl]ethyl}amino)hexyl]oxy}butyl)benzene-sulfonamide (as disclosed in WO 02/066422);
3-(3-{[7-({(2R)-2-hydroxy-2-[4-hydroxy-3-hydroxymethyl)phenyl]ethyl}-amino)heptyl]oxy}propyl)benzenesulfonamide (as disclosed in WO 02/066422);
4-{(1R)-2-[(6-{2-[(2,6-dichlorobenzyl)oxy]ethoxy}hexyl)amino]-1-hydroxyethyl}-2-(hydroxymethyl)phenol (as disclosed in WO 03/024439);
4-{(1R)-2-[(6-{4-[3-(cyclopentylsulfonyl)phenyl]butoxy}hexyl)amino]-1-hydroxyethyl}-2-(hydroxymethyl)phenol (as disclosed in WO 04/037773);
N-[2-hydroxy-5-[(1R)-1-hydroxy-2-[[2-4-[[(2R)-2-hydroxy-2-phenylethyl]amino]phenyl]ethyl]amino]ethyl]phenyl]formamide (as disclosed in WO 01/42193) and
N-{2-[4-(3-phenyl-4-methoxyphenyl)aminophenyl]ethyl}-2-hydroxy-2-(8-hydroxy-2(1H)-quinolinon-5-yl)ethylamine (as disclosed in WO 03/042160).

Where used herein the term “pharmaceutically active agent” can also be taken to include a combination containing two or more pharmaceutically active agents of the type described above. Preferred formulations containing combinations of active ingredients contain salbutamol (e.g., as the free base or the sulphate salt) salmeterol (e.g., as the xinafoate salt), formoterol (e.g. as the fumarate salt) or a long acting β₂-adrenoreceptor agonists in combination with an anti-inflammatory steroid such as a beclomethasone ester (e.g., the dipropionate), a fluticasone ester (e.g., as the propionate or 6α, 9α-difluoro-11β-hydroxy-16α-methyl-3-oxo-17α-propionyloxy-androsta-1,4-diene-17β-carbothioic acid S-(2-oxo-tetrahydro-furan-3-yl)ester), or budesonide.

A particularly preferred combination of active agents is fluticasone propionate and salmeterol, or a pharmaceutically acceptable salt thereof (particularly the xinafoate salt). Such a combination is described in patent EP0416951B1 (Glaxo Group Limited).

Further combinations of particular interest are budesonide and formoterol (e.g. as the fumarate salt) and also salmeterol, or a pharmaceutically acceptable salt thereof (particularly the xinafoate salt) and an anti-cholinergic such as ipratropium (e.g. as the bromide).

The quantity of active agent in the composition produced in accordance with this invention will vary significantly depending, inter alia, upon the particular active agent under consideration, the age and weight of the patient and the severity of the condition. Such considerations are familiar to the person skilled in the art. The active agent can be present in a concentration of 0.01-99%. Typically however, the active agent will be present in a concentration of 0.05 to 50%, more typically 0.1-15% of the total weight of the composition.

The excipient may be composed of particles of any pharmacologically inert material or combination of materials which is/are suitable for inhalation.

Preferred excipients include mono-saccharides, such as mannitol, arabinose, xylitol and dextrose and monohydrates thereof, disaccharides, such as lactose, maltose and sucrose, and polysaccharides such as starches, dextrins or dextrans. More preferred excipients comprise particulate crystalline sugars such as glucose, fructose, mannitol, sucrose and lactose. Especially preferred excipients are anhydrous lactose and lactose monohydrate.

Generally, the particle size of the majority of the excipient particles is much greater than that of the inhaled active agent and as a result, few penetrate into the respiratory tract. Thus, excipient particles for inhalable compositions may typically have particle sizes greater than 20 μm, more preferably in the range 20-150 μm. The mean geometric diameter (D(0.5)) may be in the range 20 to 150 μm, preferably in the range 25 to 90 μm, for example 65 μm.

If desired, the inhalable compositions may contain two or more excipient particle size ranges. For example, the composition may comprise two component of the excipient, the two components having different particle size distributions, a fine component and a coarse component. For example, in order to control the proportion of inhaled medicament, while retaining a good accuracy for metering, it is often desirable to use a fine component of the excipient that has a significant weight of particles (for example 10-50%, preferably 20-40%) of a size of less than 15 μm and a coarse component of the excipient which has a particle size of greater than 20 μm but lower than 150 μm, preferably lower than 100 μm. The fine particle component may have an average geometric diameter of from 15 to 50 μm. For example, the fine component may contain around 30% w/w particles of size <15 μm and have an average geometric diameter of around 30 μm. The ratio between the fine and coarse components may be adjusted depending on the application to which the formulation is to be put.

The excipient or excipients may be commercially available in the desired particle size range or may be separated by air classification, sieving or any other method of size classification known in the art.

Preferably the weight ratio of the fine and coarser excipients components will range from 1:99 to 50:50.

Fine and coarse excipient components may consist of chemically identical or chemically different substances. The excipient mixtures may, for example, contain one chemical substance as the fine excipient and a different substance as the coarser excipient. However, the fine and coarser excipients in question may themselves constitute mixtures of different substances. Preferably the fine and coarser excipients will both be lactose.

The proportion of excipient material to be used in the inhalable compositions of this invention may vary depending upon the particular active agent, the powder inhaler for administration etc. The proportion may, for example, be about 75% to 99.5% by weight of the composition as a whole.

It will be appreciated that such inhalable compositions may also contain minor amounts of other additives e.g. taste masking agents or sweeteners. It will be further appreciated that the inhalable compositions of this invention may also include yet further additives which improve stability performance, for example, magnesium stearate. Where such additives are present, they will generally not exceed 10% by weight of the total weight of the composition.

The dry powder pharmaceutical compositions in accordance with this invention can be prepared using standard methods. The pharmaceutically active agent, excipient and calcium stearate can be intimately mixed using any suitable blending apparatus, such as high shear blenders. The particular components of the formulation can be admixed in any order. Pre-mixing of particular components may be found to be advantageous in certain circumstances. The progress of the blending process can be monitored by carrying out content uniformity determinations. For example, the blending apparatus may be stopped, materials removed using a sample thief and then analysed for homogeneity by High Performance Liquid Chromatography (HPLC).

To determine the improved stability associated with compositions prepared according to this invention, the blends thus formed can be placed on accelerated stability screen (e.g. 40° C./75% relative humidity) and the fine particle fraction reduction (i.e. comparison of pre and post stability FPF data) measured as an analytical parameter using a Cascade Impactor (CI) or Twin Stage Impinger (TSI). Such procedures are familiar to those skilled in the art.

According to the invention, the inhalable compositions can be delivered by any suitable inhalation device that is adapted to administer a controlled amount of such a pharmaceutical composition to a patient. Suitable inhalation devices may rely upon the aerosolisation energy of the patient's own breath to expel and disperse the dry powder dose. Alternatively, this energy may be provided by an energy source independent of the patient's inhalation effort, such as by impellers, patient/device created pressurised gas sources or physically (e.g. compressed gas) or chemically stored energy sources.

Suitable inhalation devices can also be of the reservoir type i.e. where the dose is withdrawn from a storage vessel using a suitably designed dosing device or alternatively, inhalation devices that release drug from pre-metered units e.g. blisters, cartridges or capsules.

Packaging of the composition may be suitable for unit dose or multi-dose delivery. In the case of multi-dose delivery, the composition can be pre-metered (e.g. Diskhaler® as described in U.S. Pat. No. 4,811,731 and U.S. Pat. No. 5,035,237) or metered in use (e.g. Turbuhaler® as described in U.S. Pat. No. 4,668,218). An example of a unit-dose device is Rotahaler® (as described in U.S. Pat. No. 4,353,365).

A particularly preferred inhalation device for dry powder pharmaceutical compositions of this invention is the Diskus® inhaler (described in U.S. Pat. Nos. 5,590,645 and 5,860,149) which may be charged with blister (medicament) packs as described in U.S. Pat. No. 5,873,360. The drawings of said United States patents are specifically incorporated by reference.

The present invention therefore also provides for a medicament pack for use in an inhalation device which comprises an elongate strip formed from a base sheet having a plurality of recesses spaced along its length and a lid sheet hermetically but peelably sealed thereto to define a plurality of containers, each container having therein an inhalable composition according to the present invention.

Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

As a yet further aspect of the present invention we also provide an inhalation device for use with a medicament pack which comprises an inhalable composition according to the present invention, said device comprising:
(i) an opening station for receiving a container of a medicament pack being used with said inhalation device;
(ii) means positioned to engage peelable sheets of a container which has been received in said opening station for peeling apart the peelable sheets, to open such a container;
(iii) an outlet, positioned to be in communication with an opened container, through which a user can inhale medicament in powder form from such an opened container; and
(iv) indexing means for indexing in communication with said outlet containers of a medicament pack in use with said inhalation device.

As an alternative aspect of the present invention we also provide a medicament pack comprising a circular carrier disc which has a plurality of pre-filled, hermetically sealed containers formed integrally therewith and arranged in a circle, each container containing an inhalable composition according to the present invention, each container being puncturable to form a hole on each side thereof to allow in use, air to flow through the container to entrain the powder contained therein.

As a further aspect of the present invention there is also provided an inhalation device by which compositions of the present invention may be administered to a patient which comprises a housing, a tray mounted and capable of moving within said housing (via a plunger) adapted to receive a circular carrier disc medicament pack, an air inlet (through which air can enter said device) and an air outlet (through which a patient may inhale and receive said composition.

As an alternative aspect of the present invention we also provide a medicament pack comprising a piercable capsule which contains an inhalable composition according to the present invention.

As a further aspect of the present invention there is also provided an inhalation device by which compositions of the present invention may be administered to a patient which comprises a body shell which has a nozzle at a forward end and which is open at the rear end, a sleeve fitted on the outside of the body shell and rotatable with respect to it, a means for retaining a piercable capsule extending through the rear wall of the sleeve into the body shell, means for piercing said capsule when sleeve is rotated and a guard to ensure that the inhalable composition and not the pierced capsule, passes through the nozzle.

As a further aspect of the present invention there is also provided an inhalation device by which inhalable compositions of the present invention may be administered to a patient which comprises a nozzle, an air conduit connected to said nozzle for allowing a passage of air to be inhaled, a dosing unit comprising a storage chamber for the inhalable composition (which may also comprise a dosage indicating means) and a displaceable element for dispensing said formulation from the storage chamber into the air conduit, a maneuvering unit for displacing said element in relation to the storage chamber and optional deflector devices to provide accelerated airflow.

In a further or alternative aspect the present invention also provides for a method of treatment or prophylaxis of respiratory disorders which comprises administering to a patient in need thereof of a dry powder pharmaceutical composition according to the present invention.

According to another aspect the present invention provides for the use of a dry powder pharmaceutical composition according to the present invention in the manufacture of a medicament for the treatment of respiratory disorders.

Suitable examples of respiratory disorders include, but are not limited to, asthma, bronchitis, chronic obstructive pulmonary disease (COPD), emphysema and rhinitis.

Preferably the respiratory disorder is asthma.

Where used herein, unless otherwise stated, the terms “dry powder pharmaceutical composition for inhalation therapy” and “inhalable composition” are to be treated as synonymous.

All publications, including but not limited to patents and patent applications, cited in this specification are herein incorporated by reference as if each individual publication were specifically and individually indicated to be incorporated by reference herein as though fully set forth.

Throughout the specification and claims which follow, unless the context requires otherwise, the word “comprise”, and variations thereof such as “comprises” and “comprising”, will be understood to imply the inclusion of a stated integer or step or group of integers but not to the exclusion of any other integer or step or groups of integers.

The invention will now be described in detail by way of reference only to the following non-limiting examples.

### EXAMPLE 1

3 Day and 1 Month FPF Stability Study for Dry Powder Compositions Comprising Calcium Stearate and

a) Compound X 10 μg (Base Equivalent)

b) Salmeterol (as Xinafoate) 50 μg, Ipratropium Bromide 160 μg

Calcium stearate was obtained from Whittaker, Clark and Daniels (South Plainfield, N.J., USA). Lactose was supplied by Borculo Domo Ingredients, Netherlands.

The blends A, B, C and D as tabulated below, were prepared by the following procedure.

Compound X is the maleate salt of the compound of example 11 described in WO98/28319. Blends A and C, the controls, were formed by mixing lactose with Compound X and Salmeterol xinafoate/Ipratropium bromide respectively using a high shear blender for approximately 10 minutes (blend uniformity less than 4% RSD for either active material (ten samples each approx. 25 mg)).

For blends B and D, the calcium stearate was pre-mixed with the lactose in a high shear blender. The active agents were then added and blending was performed for 10 to 15 minutes. The blend uniformity data were found to be in the range 1-4% RSD for both active materials.

TABLE 1

| Blend | Contents of blend | Amount (g) | Amount (%) |
|---|---|---|---|
| A | Compound X (micronised) | 0.50 | 0.10 |
| | Lactose monohydrate 16% fines** | 499.50 | 99.92 |
| B | Compound X (micronised) | 0.50 | 0.10 |
| | Lactose monohydrate 16% fines** | 492.00 | 98.40 |
| | Calcium stearate D(0.5) 3.6 μm** | 7.50 | 1.50 |
| C | Salmeterol xinafoate (micronised) | 6.96 | 0.58 |
| | Ipratropium Bromide (micronised) | 16.05 | 1.33 |
| | Lactose monohydrate 8-10% fines* | 1177.20 | 98.08 |
| D | Salmeterol xinafoate (micronised) | 6.98 | 0.58 |
| | Ipratropium Bromide (micronised) | 16.05 | 1.33 |
| | Lactose monohydrate 8-10% fines* | 1158.9 | 96.6 |
| | Calcium stearate D(0.5) 3.6 μm** | 18.05 | 1.50 |

*Laser diffraction using Malvern Mastersizer, sample dispersed in lecithin/Isooctane
**Laser diffraction using Sympatec, Vibri sample introduction at 1 bar pressure
(Fines = material < 15 μm)

The blends thus formed were then added to blister packs, of the type described in patent U.S. Pat. No. 5,873,360, using filling methods according to procedures outlined in WO 00/71419 (Glaxo Group Limited). Each blister contained approximately 12.5 mg of the blend.

For rapid screening, the pockets in one portion of the blister packs were then pierced with a 0.75 mm pin and the blister packs were loaded into a Diskus® device.

The loaded Diskus® devices containing blends A, B, C and D were placed in accelerated stability test environment at 40° C./75% relative humidity for period of 72 hours for A and B and 48 hours for C and D.

For longer term screening, another set of blister packs was loaded into a Diskus® device without piercing. Those Diskus® devices containing blends A, B, C and D were placed in accelerated stability test environment at 40° C./75% relative humidity for period of one month.

Twin stage impinger analysis was performed (at 60 l/min) on the salmeterol xinafoate/ipratropium bromide sample C before storage and samples C and after storage by the method detailed in the British Pharmacopoeia (Method A) with the exception that a USP throat was substituted for the glass one and was sealed to the stage 1 jet tube using a rubber gasket. The devices were tested pre and post storage by discharging the contents of 14 blisters into the Twin Stage Impinger apparatus. The results obtained are tabulated below in Table 2.

Similar testing was performed for the compound X samples (blends A and B) and the bland D initial time point sample, with the exception that an Andersen cascade impactor was used at 60 l/min flow rate. Impaction stages 2, 3, 4, 5, 6 and 7 were not used. The deposition in stage 1 and in the filter stage represents the fine particle dose.

TABLE 2

| | % Fine Particle Fraction | | |
|---|---|---|---|
| Blend | Pre-Storage | Rapid screen | 1 month |
| A | 23.8 | 10.3 | 12.3 |
| B | 43.5 | 39.2 | 44.5 |
| C Salmeterol | 24.7 | 8.0 | 12.5 |
| C Ipratropium bromide | 39.2 | 13.3 | 20 |
| D Salmeterol | 33.8 | 26.2 | 26.1 |
| D Ipratropium bromide | 44.5 | 32.5 | 36.1 |

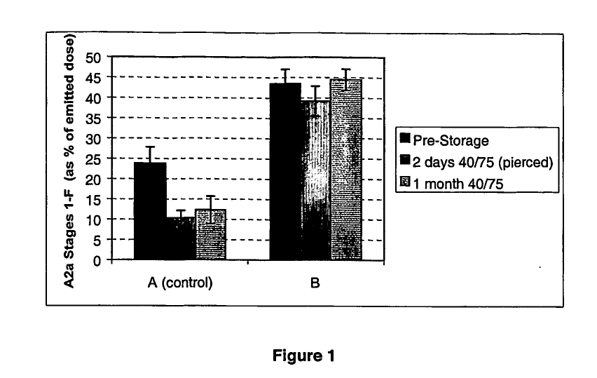

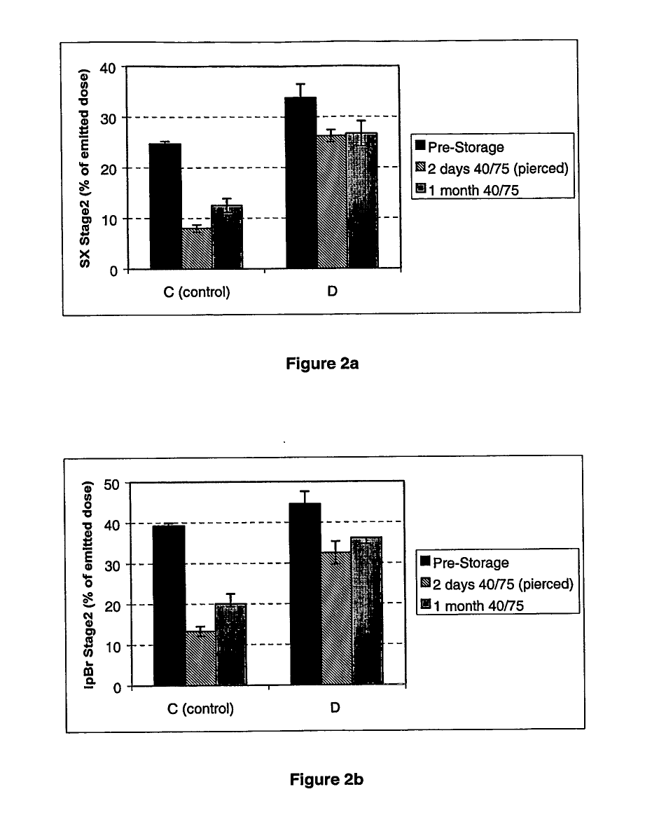

These data are represented graphically in FIGS. 1 and 2.

FIG. 1 shows the effect of calcium stearate on the cascade impactor performance of the compound X formulations.

FIGS. 2a and 2b show the effect of calcium stearate on the twin impinger performance of the salmeterol and ipratropium bromide components respectively of the Blend C and D formulations.

### EXAMPLE 2

12 Month FPF Stability Study for Dry Powder Compositions Comprising Calcium Stearate, Salmeterol Xinafoate 50 μg (Base Equivalent) and Fluticasone Propionate 50 μg

Calcium stearate and lactose were obtained and used as described above in Example 1. Blends E and F, as tabulated below, were prepared by the following procedure.

Blend E, the control, was formed by mixing lactose with salmeterol xinafoate and fluticasone propionate in a 2.5 L QMM (high shear) bowl for approximately 10 minutes (blend uniformity less than 4% RSD for either active material (ten samples each approx. 25 mg)).

For blend F the calcium stearate was pre-mixed with the lactose for 7 minutes. The salmeterol xinafoate and fluticasone propionate were then added and the mixture blended for 13 minutes. The blend uniformity data for the salmeterol xinafoate gave as 20% RSD due to one high result, the fluticasone propionate blend uniformity gave a 4% RSD.

TABLE 3

| Blend | Contents of blend | Amount (g) | Amount (%) |
|---|---|---|---|
| E | Salmeterol xinafoate (micronised) | 2.91 | 0.58 |
| | Fluticasone propionate (micronised) | 2.00 | 0.40 |
| | Lactose monohydrate 8.7% fines* | 495.00 | 99.02 |
| F | Salmeterol xinafoate (micronised) | 2.90 | 0.58 |
| | Fluticasone propionate (micronised) | 2.00 | 0.40 |
| | Calcium stearate D (0.5) 3.6 μm** | 7.51 | 1.5 |
| | Lactose monohydrate 8.7% fines* | 497.81 | 97.52 |

*Laser diffraction using Malvern Mastersizer, sample dispersed in lecithin/Isooctane (Fines = material < 15 μm)
**Laser diffraction using Sympatec, Vibri sample introduction at 1 bar pressure

The blends thus formed were then added to blister packs, of the type described in patent U.S. Pat. No. 5,873,360, using filling methods according to procedures outlined in WO 00/71419 (Glaxo Group Limited). Each blister contained approximately 12.5 mg of the blend.

For screening, each blister pack was loaded into a Diskus® device. The Diskus devices containing blends E and F were placed in an accelerated stability test environment at 40° C./75% relative humidity for period of twelve months.

Twin stage impinger analysis was performed (at 60 l/min) on each of the sample types before and after storage by the method detailed in the British Pharmacopoeia (Method A) with the exception that a USP throat was substituted for the glass one and was sealed to the stage 1 jet tube using a rubber gasket. The devices were tested pre and post storage by discharging the contents of 10 or 14 blisters into the Twin Stage Impinger apparatus. The results obtained are tabulated below in Table 4.

TABLE 4

% Fine Particle Fraction

| Blend | Pre-Storage | 12 month storage |
|---|---|---|
| E - salmeterol xinafoate | 23.0 | 6.0 |
| F - salmeterol xinafoate | 38.6 | 23.2 |
| E - fluticasone propionate | 28.7 | 6.0 |
| F - fluticasone propionate | 38.3 | 23.6 |

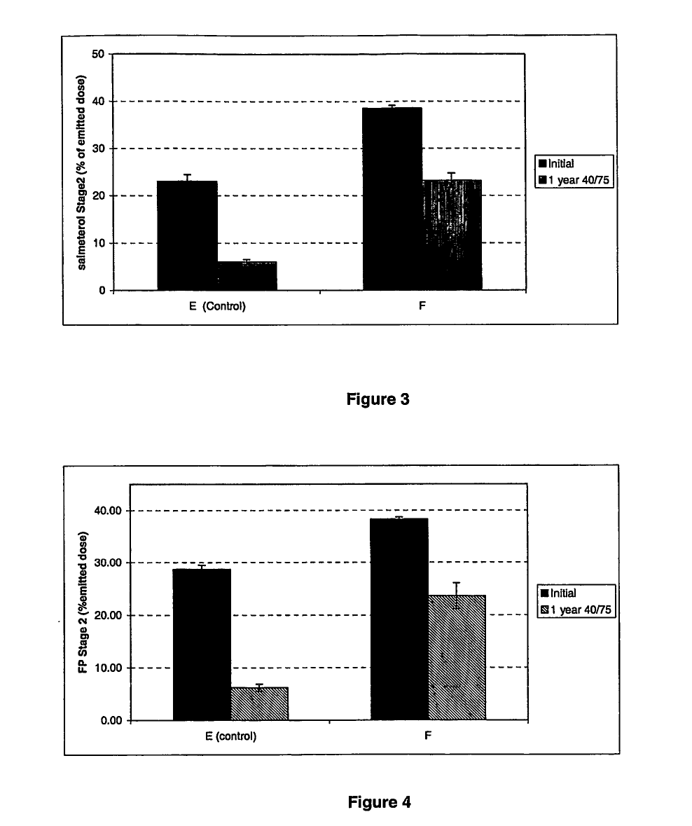

These data are represented graphically in FIGS. 3 and 4.

FIG. 3 shows the effect of calcium stearate on the twin impinger performance of the salmeterol component of the formulations.

FIG. 4 shows the effect of calcium stearate on the twin impinger performance of the fluticasone propionate component of the formulations.

### EXAMPLE 3

FPM Studies on Dry Powder Formulations Comprising 0.1% w/w Compound Y and Varying Concentrations of Calcium Stearate

Compound Y is the cinnamate salt of 3-(4-{[6-({(2R)-2-hydroxy-2-[4-hydroxy-3-(hydroxymethyl)-phenyl]ethyl}amino)hexyl]oxy}butyl)benzene-sulfonamide. This compound can be prepared using methods described in patent application WO 02/066422.

Calcium stearate (Liga CPR-2-V Pharmaceutical Grade) was obtained from Peter Greven (Netherlands). Lactose monohydrate containing 6% fines (as defined as the fraction of particles below 15 μm) was supplied by Borculo Domo Ingredients (Netherlands).

Blends G-K as described in Table 5 below, were prepared on a 500 g scale using a high shear blender, whereby the calcium stearate was premixed with the lactose for approximately 10 minutes, and then blended with Compound Y for the same time period. All blends were shown to be homogenous with respect to uniformity of the active ingredient (RSD<3%). The blends were filled into MDPI foil strips (see e.g., U.S. Pat. No. 5,860,419) using perforated bed filling methodology and the FPM measured from the Diskus® device using Andersen Cascade Impaction at 60 l/min.

TABLE 5

| Blend | Calcium stearate (% w/w) |
|---|---|
| G | — |
| H | 0.5 |
| I | 1.0 |
| J | 2.0 |
| K | 4.0 |

### EXAMPLE 3A

### Initial FPM Measurement

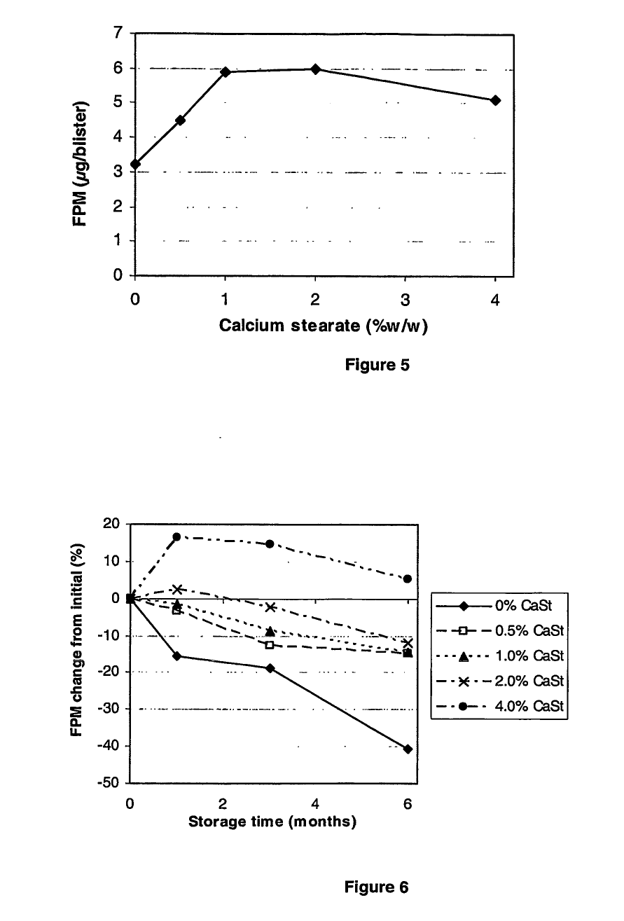

FIG. 5 shows the initial FPM of dry powder formulations containing 0.1% w/w Compound Y and varying concentrations of calcium stearate (CaSt).

The results illustrated in FIG. 5 show that all formulations containing calcium stearate (Blends H-K) had a higher FPM relative to the lactose only formulation (Blend G).

### EXAMPLE 3

### FPM Stability Study

MDPI foil strips containing blends G-K as described in Table 5 above were placed on stability at 30° C./65% RH and 40° C./75% RH. FPM was measured from Diskus devices using Andersen Cascade Impaction at 60 l/min (n=3) following storage up to 6 months.

FIG. 6 shows the FPM of dry powder formulations containing 0.1% w/w Compound Y and varying concentrations of calcium stearate (CaSt) relative to initial following storage at 30° C./65% RH

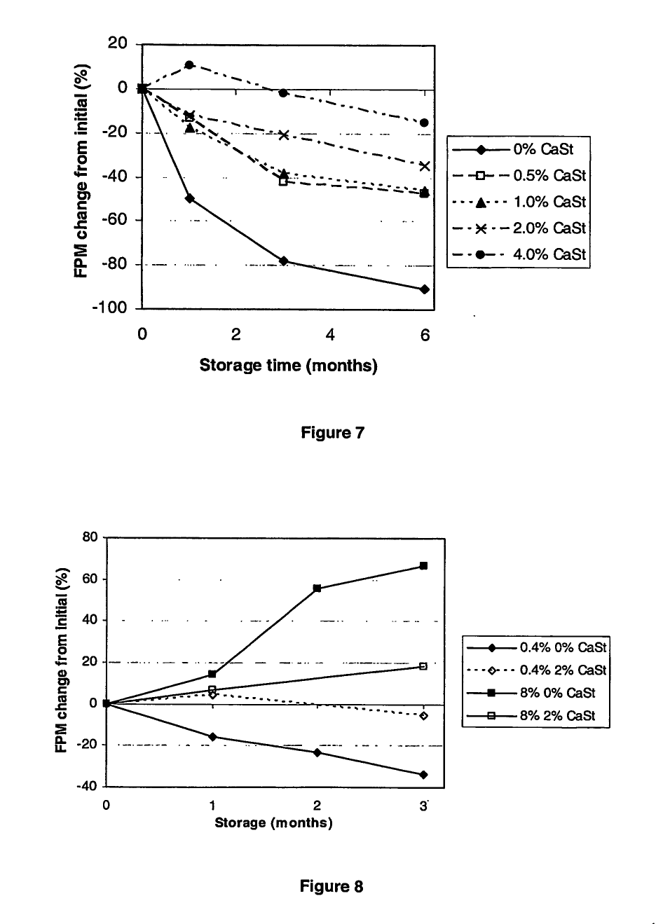

FIG. 7 shows the FPM of dry powder formulations containing 0.1% w/w Compound Y and varying concentrations of calcium stearate (CaSt) relative to initial following storage at 40° C./75% RH.

The results illustrated in FIGS. 6 and 7 showed a significant improvement in FPM at both stability conditions for dry powder formulations containing calcium stearate (Blends H-K) in comparison with the lactose only formulation (Blend G). Greater reductions on FPM drop are seen with increasing concentrations of calcium stearate.

### EXAMPLE 4

3 Months FPM Stability Study for Dry Powder Compositions Comprising 0.4 and 8% w/w of Compound Z and 2% Calcium Stearate

Compound Z is 6α, 9α-Difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl. This compound can be prepared using methods described in patent application WO 02/12265.

Calcium stearate was used as described in Example 3. Lactose monohydrate containing 8% fines was sourced as described in Example 3.

Blends L-O described in Table 6 below were manufactured on a 300 g scale using a high shear blender, whereby the calcium stearate was premixed with the lactose for approximately 10 minutes, and then blended with Compound Z for the same time period. All blends were shown to be homogenous with respect to uniformity of the active ingredient (RSD<3%). The blends were filled into MDPI foil strips (see e.g., U.S. Pat. No. 5,860,419) using perforated bed filling methodology and the FPM measured pre and post storage periods at 40° C./75% RH. FPM was measured from Diskus device using Andersen Cascade Impaction at 60 l/min (n=3).

TABLE 6

| Blend | Compound Y (% w/w) | Calcium stearate (% w/w) |
|---|---|---|
| L | 0.4 | — |
| M | 0.4 | 2 |
| N | 8 | — |
| O | 8 | 2 |

FIG. 8 shows the FPM of dry powder formulations containing 0.4 and 8% w/w Compound Z formulations following storage at 40 C/75% RH

The data shown in the Examples 1-4 demonstrate that the incorporation of calcium stearate in a dry powder pharmaceutical composition significantly increases the fine particle fraction/mass emitted from a DPI device.

Furthermore, the data also show the incorporation of calcium stearate in a dry powder pharmaceutical composition significantly reduces the deterioration in fine particle fraction/mass following exposure to high temperature and humidity. It is believed therefore, that such compositions, when incorporated in dry powder inhaler products, would demonstrate improved performance and/or considerably enhanced stability and hence an increased shelf-life.

Without wishing to be bound by this theory, we believe that conventional dry powder blends (e.g. those containing an active agent and excipient such as lactose), when subject to environmental humidity, result in a liquid film forming on the fine lactose particles (<15 μm) which allows dissolution of the lactose. When the humidity decreases, the lactose solution evaporates allowing the formation of permanent crystal bridges between the lactose particles and between the active agent and fine lactose particles. The resultant active agent/lactose agglomerates are not readily aerosolised and cause a reduction in the fine particle fraction. The addition of calcium stearate dispersed in the blend with active agent and the lactose particles may therefore allow the lactose to be coated with calcium stearate particles which being insoluble and hydrophobic prevent dissolution of the lactose and hence prevent the formation of the crystal bridges between the fine lactose particles and active agent particles, hence reducing agglomeration and the consequent decline in fine particle fraction. It also appears that adhesion between lactose particles and particles of active are reduced by the presence of calcium stearate dispersed in the blend. That appears to result in an increase in fine particle fraction even before storage.

What is claimed is:

1.-3. (canceled)

4. A dry powder pharmaceutical composition for inhalation therapy comprising a pharmaceutically active agent, an excipient and calcium stearate.

5. A dry powder composition according to claim 4 in which the calcium stearate is in particulate form.

6. A dry powder composition according to claim 4 in which the calcium stearate is a hydrate.

7. A dry powder composition according to claim 4 in which the calcium stearate is anhydrous.

8. A dry powder pharmaceutical composition according to claim 4 in which the calcium stearate is present at a concentration of from 1 to 10% of the total composition.

9. A dry powder pharmaceutical composition according to claim 4 in which the calcium stearate has an geometric size in the range 1-20 μm.

10. A dry powder pharmaceutical composition according to claim 4 which comprises two component of the excipient, the two components having different particle size distributions.

11. A dry powder pharmaceutical composition according to claim 10 in which the fine and coarse excipient components are both lactose.

12. A dry powder pharmaceutical composition according to claim 4 in which the pharmaceutically active agent is a combination of fluticasone propionate and salmeterol or a pharmaceutically acceptable salt thereof.

13. A dry powder pharmaceutical composition according to claim 4 in which the pharmaceutically active agent is 3-(4-{[6-({(2R)-2-hydroxy-2-[4-hydroxy-3-(hydroxymethyl)-phenyl]ethyl}amino)hexyl]oxy}butyl)benzene-sulfonamide and/or 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester.

14. A method of increasing fine particle dose performance for an active ingredient substance in a dry powder pharmaceutical composition for inhalation therapy comprising a pharmaceutically active agent and an excipient, which comprises mixing calcium stearate with said active ingredient substance and said carrier.

15. A method of improving stability performance of an active ingredient substance in a formulation comprising a carrier and an active ingredient substance, which method comprises mixing calcium stearate with said active ingredient substance and said carrier.

16. A method of eliminating or reducing the detrimental effect on fine particle dose of an active ingredient substance caused on storage in a formulation comprising a carrier and the active ingredient substance, which method comprises mixing calcium stearate with said active ingredient substance and said carrier.

17. A method as claimed in claim 14 further comprising one or more of the features described in claim 4.

18. A method of treatment or prophylaxis of respiratory disorders which comprises administering to a patient in need thereof a dry powder pharmaceutical composition according to claim 4.

19. (canceled)

20. An inhalation device containing therein a dry powder pharmaceutical composition according to claim 4.

21. An inhalation device according to claim 20 in which the dry powder pharmaceutical composition is released from a pre-metered unit medicament pack.

22. A medicament pack for use in an inhalation device which comprises an elongate strip formed from a base sheet having a plurality of recesses spaced along its length and a lid sheet hermetically but peelably sealed thereto to define a plurality of containers, each container having therein an inhalable composition according to claim 4.

23. A medicament pack according to claim 22 wherein the strip is sufficiently flexible to be wound into a roll.

24. A medicament pack according to claim 22 wherein the lid sheet and base sheet have leading end portions which are not sealed to one another.

25. A medicament pack according to claim 24 wherein at least one of the said leading end portions is constructed to be attached to a winding means.

26. A medicament pack according to claim 22 wherein the hermetic seal between the base and lid sheets extends over their whole width.

27. A medicament pack according to claim 22 wherein the lid sheet may be peeled from the base sheet in a longitudinal direction from a first end of the said base sheet.

28. An inhalation device for use with a medicament pack according to claim 22 which comprises an inhalable composition according to claim 4, said device comprising:
(i) an opening station for receiving a container of a medicament pack being used with said inhalation device;
(ii) means positioned to engage peelable sheets of a container which has been received in said opening station for peeling apart the peelable sheets, to open such a container;
(iii) an outlet, positioned to be in communication with an opened container, through which a user can inhale medicament in powder form from such an opened container; and
(iv) indexing means for indexing in communication with said outlet containers of a medicament pack in use with said inhalation device.

29. A medicament pack comprising a circular carrier disc which has a plurality of pre-filled, hermetically sealed containers formed integrally therewith and arranged in a circle, each container containing an inhalable composition according to claim 4, each container being puncturable to form a hole on each side thereof to allow in use, air to flow through the container to entrain the powder contained therein.

30. An inhalation device by which inhalable compositions according to claim 4 may be administered to a patient which comprises a housing, a tray mounted and capable of moving within said housing (via a plunger) adapted to receive a circular carrier disc medicament pack according to claim 29, an air inlet (through which air can enter said device) and an air outlet (through which a patient may inhale and receive said composition.

31. A medicament pack comprising a piercable capsule which contains an inhalable composition according to claim 4.

32. An inhalation device by which inhalable compositions according to claim 4 may be administered to a patient which comprises a body shell which has a nozzle at a forward end and which is open at the rear end, a sleeve fitted on the outside of the body shell and rotatable with respect to it, a means for retaining a piercable capsule according to claim 31 extending through the rear wall of the sleeve into the body shell, means for piercing said capsule when sleeve is rotated and a guard to ensure that the composition and not the pierced capsule, passes through the nozzle.

33. An inhalation device by which inhalable compositions according to claim 4 may be administered to a patient which comprises a nozzle, an air conduit connected to said nozzle for allowing a passage of air to be inhaled, a dosing unit comprising a storage chamber for the composition (which may also comprise a dosage indicating means) and a displaceable element for dispensing said composition from the storage chamber into the air conduit, a manoeuvring unit for displacing said element in relation to the storage chamber and optional deflector devices to provide accelerated airflow.
